(19) United States
(12) Patent Application Publication
(10) Pub. No.: US 2020/0273568 A1
(43) Pub. Date: Aug. 27, 2020

(54) Method For Operating At Least One Respirator In A Communication Network

(71) Applicant: IMT Analytics AG, Buchs SG (CH)

(72) Inventors: Harri FRIBERG, Mauren (LI); Jakob DAESCHER, Fläsch (CH)

(73) Assignee: IMT Analytics AG, Buchs SG (CH)

(21) Appl. No.: 16/494,943

(22) PCT Filed: Mar. 20, 2018
(86) PCT No.: PCT/IB2018/051862

§ 371 Date: Sep. 17, 2019

(30) Foreign Application Priority Data
Mar. 21, 2017 (EP) 17162135.2

(51) Int. Cl. G16H 40/67; H04L 29/08; A61M 16/00; G16H 50/20; G16H 40/40; G16H 20/40; G16H 50/70; H04L 29/06

(52) U.S. Cl. CPC G16H 40/67; H04L 67/12; A61M 16/024; G16H 50/20; G16H 40/40; A61M 2205/18; G16H 50/70; H04L 63/10; A61M 2205/50; A61M 2205/3553; A61M 2205/502; G16H 20/40

(57) ABSTRACT

Methods and apparatus provide for operating at least one respirator in a communication network, where the communication network (II) has at least one processing unit, a cloud, and the at least one processing unit has a computing unit, and the at least one respirator is connected to the communication network via a communication module. The respirator data from the at least one respirator is transmitted to the communication module and then the transmitted respirator data is sent from the communication module to the at least one processing unit. The sent respirator data is analyzed in the at least one processing unit, the analyzed respirator data is revised in the computing unit and then transferred from the at least one processing unit to a communication module and/or from the at least one processing unit to at least one external receiving unit.

### BACKGROUND

The invention relates to a method for operating at least one respirator in a communication network.

A communication network comprises an infrastructure consisting of a plurality of units for the transmission of information and data. Communication networks also represent data connections between a plurality of terminals. An important pre-requisite for communication networks is the standardisation of the interfaces and the logic functions. This is achieved by the orientation towards hierarchically constructed architectural models with a plurality of standardised protocol levels. The integration of medical devices into a communication network requires a high degree of reliability in the communication network, in order that the medical devices can also be operated therein in an error-free manner. The safety of ventilated patients depended decisively hitherto on the affordable control pattern of the support staff. Close-knit patient-monitoring and therefore an improvement in patient safety could only be achieved through much higher support costs. In the case of an acute threat to the ventilated patient, the user responsible, e.g. a doctor, had to travel immediately to the hospital to make changes to the adjustments of the respirator.

WO 2013/002650 A1 discloses a computer system for relaying compliance data and other information from respirators to external interested parties. The respirator data are formatted in a processor and summarised into a report. The respirator data are then sent via a network to external interested parties. The network comprises a reminder server, which sends standardised reminder messages to the external interested parties.

A drawback with this known solution is that the sent respirator data cannot already be analysed in the computer system, but rather are sent to external interested parties by means of formatted data reports and then filed there in databases. The data reports are then merely presented to the different terminals.

US 2016/0206838 A1 discloses a monitoring system with a plurality of respirators, which comprises a memory and a processor. Instructions and data are stored in the memory, which after transfer to the processor are processed by the latter. The processor analyses the data of the plurality of respirators and transmits these analysed data, for example onto a display of the respirator or to a receiving unit of a member of the medical staff. US 2013/U199533 A1 shows a comparable monitoring system.

A drawback with this known solution is that the data are analysed and prepared for the medical staff in reports or recommendations for treatment, so that the medical staff are absolutely essential for the operation of the plurality of respirators in the monitoring system.

### SUMMARY

The problem of the present invention, therefore, is to create a method for operating at least one respirator in a communication network, which does not have the aforementioned drawbacks, and in particular to monitor and influence in real-time the operational behaviour of the at least one respirator. It is also the problem of the invention to provide a computer program product for such a method.

The problem is solved by the features of the independent claim. Advantageous developments are presented in the figures and in the dependent claims.

The method according to the invention for operating at least one respirator in a communication network, wherein the communication network comprises at least one processing unit, advantageously a cloud, and the at least one processing unit comprises a computing unit and the at least one respirator is connected via a communication module to the communication network, is characterised by the following steps.

Transmission of respirator data from at least one respirator to the communication module (step a)) and subsequent sending of the transmitted respirator data from the communication module to the at least one processing unit (step b)).

The transmitted respirator data are advantageously sent from the communication module to the at least one processing unit via a GSM-standard, so that a rapid and simple transmission in a GSM network takes place. As an alternative to this, other transmission standards, such as for example Bluetooth® or Wireless LAN, are also possible, wherein the selection of the transmission standard is advantageously adapted to the regional location of the respirator.

The sent respirator data are then analysed in the least one processing unit (step c)) and the analysed respirator data are revised in the computing unit of the least one processing unit (step d)). The analysed and revised respirator data are then transmitted from the at least one processing unit to a communication module (step e)).

The transmitted respirator data are then used (step f)).

The central analysis of the respirator data in the at least one processing unit enables a comprehensive pooling of respirator data, wherein, when they are used, already interlinked information and, where applicable, information of different types of device are prepared with the aid of the communication unit. The transmitted respirator data can be used for the direct control of the at least one respirator and/or as information or as further training for a user, as a scientific study for a user or as input parameters for the patient of the at least one respirator.

In this connection, the analysis of respirator data corresponds not only to the mere merging or listing of data points, but also to the precise examination and validation of the plausibility of individual respirator data points. Moreover, an analysis of curves of a plurality of respirator data points and an analysis of mutual dependencies of different respirator data points is thus also included. The respirator data are parameters which, on the one hand, result from the active or inactive ventilation of the patient (ventilation parameters such as ventilation mode, type of hose system, alarm logbook, event logbook, all ventilation adjustment parameters, all ventilation measurement values such as inhalation and exhalation pressure, ventilation frequency, breathing resistance, lung stiffness, compliance data, etc.), or also parameters which relate to the mechanical or electrical operating parameters of the at least one respirator itself (operating hours, maintenance intervals, battery operating hours, operating voltage, performance parameters of the ventilator etc.). Also numbering among the respirator data are parameters which include the location coordinates, ambient conditions at the location of the at least one respirator (for example air pressure, air humidity, temperature etc.), service parameters (annual service, operating hours, etc.) as well as patient and therapy data (e.g. symptoms, age of patient, duration of ventilation, medication and nutrition plan).

The revision of respirator data includes targeted changing of one or more respirator data points or respirator data point curves. The revision takes place in particular in the computing unit of the at least one processing unit in the communication network. The further revision takes place in particular in an external receiving unit or in the computing unit of the at least one processing unit.

The units communicating with one another in this communication network are for example processing units, which use the communication network in order to use storage spaces, computing power or various application software in the communication network. At least one of the processing units is constituted as a cloud and its IT structure can thus be used as a service.

Alternatively, the analysed and revised respirator data are transmitted by the at least one processing unit to at least one external receiving unit (step e)).

Alternatively, the analysed and revised respirator data are transmitted by the at least one processing unit to a communication module and the analysed and revised respirator data are transmitted by the at least one processing unit to at least one external receiving unit.

The previously mentioned communication module is advantageously the communication module of the at least one respirator, so that the analysed and revised respirator data can simply be assigned to the at least one respirator. Previously analysed and revised respirator data from the at least one respirator can thus be used in parallel by many users and a rapid reaction by the user, for example to malfunctions in at least one respirator, is possible. For this, the user solely requires an external receiving unit, which makes the analysed and revised respirator data available to the user.

The transmitted respirator data are advantageously used solely in a confined area. The transmitted respirator data thus remain for example inside the hospital, so that increased data security for the respirator data is enabled.

The analysis and revision of the respirator data in the computing unit of the at least one processing unit advantageously includes the detection of respirator data which indicates faulty operation of the respirator by the user or the patient. The computing unit of the at least one processing unit automatically detects a faulty operation of the respirator by the user or by the patient and the faulty respirator data resulting therefrom. A faulty ventilation of the patient can thus be detected in good time and the effect on the patient resulting therefrom can be prevented.

The analysed and revised respirator data are preferably transmitted from the communication module of the at least one respirator to the at least one respirator (according to step d)), so that the latter can be used in the respirator and the respirator performance of this respirator is thus improved.

The respirator data sent from the communication module are preferably stored in the at least one processing unit (before step d)), for which purpose the at least one processing unit comprises at least one memory. The stored respirator data can thus be archived and subsequently used for analysis purposes and revision purposes.

The sent respirator data are advantageously stored sorted according to their medical and/or safety-related relevance, wherein the sorting enables a structured acquisition of the sent respirator data. The further use of the sent respirator data for different purposes is thus possible. For example, the sent respirator data are made available to various users for research purposes and learning purposes. Medically relevant respirator data include those data which are related to the active or inactive ventilation of a patient and safety-relevant respirator data are those data which include mechanical or electrical operating parameters of the at least one respirator.

The sent respirator data are also advantageously stored in different memories, so that for example different access authorisations can be assigned to the sent respirator data.

The sent respirator data are advantageously stored in different memories, wherein the respirator data are stored separately according to the location of the at least one respirator and according to the medical relevance of the sent respirator data. The medical relevance of the respirator data can thus easily be compared with the location of the respirator and, in case of emergency, a rapid implementation of medical emergency measures is enabled on site.

The sent respirator data are also advantageously stored in different memories depending on the patient data (age, height, BMI, etc.), so that for example different patient data on the at least one respirator can be compared.

The analysed and revised respirator data are preferably classified into at least three categories. The analysed and revised respirator data can thus be easily and unequivocally distinguished from one another.

The classification of the analysed and revised respirator data advantageously takes place depending on their medical and/or safety-related relevance, so that the classified respirator data can easily be assigned to a warning system.

The analysed and revised respirator data are advantageously transmitted to at least one external receiving unit, so that an external user can gain access to the classified respirator data.

The classified respirator data are advantageously stored in one of the memories of the at least one processing unit. The revised respirator data can thus easily be distinguished from one another and used again at a later time.

The transmitted respirator data are preferably previously processed by the at least one communication module for secure sending to the at least one processing unit, wherein the transmitted respirator data can be simply standardised therein and the at least one processing unit can operate with standardised respirator data. For example, respirator data of respirators from different device manufacturers can thus be compared, since data formats known only to this at least one processing unit can be used in the at least one processing unit.

The transmitted respirator data are advantageously previously encoded by the at least one communication module for secure sending to the at least one processing unit, so that the data security of the sent respirator data is guaranteed.

The transmitted respirator data are advantageously processed by the at least one communication module for the secure transmission to the respirator, wherein the transmitted respirator data are simply standardised therein. Moreover, the transmitted respirator data can thus be used independently of the device manufacturer of the respirator.

The transmitted respirator data are advantageously previously decoded by the at least one communication module for the secure transmission to the respirator. The transmitted respirator data can thus easily be used in this respirator. Alternatively, a respirator with its own decoder could be used for decoding the transmitted respirator data.

The revision of the analysed respirator data in the computing unit preferably takes place with the aid of at least one computing algorithm (step d)). The analysed respirator data are then revised quickly and reliably and can be used directly.

The revision of the analysed respirator data advantageously takes place with at least one self-learning computing algorithm. The at least one computing algorithm is thus repeatedly updated and the revision of the analysed respirator data in the computing unit is thus constantly improved. The quality of the revised respirator data is thus raised, so that the revised respirator data can be used directly on the at least one respirator. The at least one self-learning computing algorithm is based on at least one artificial intelligence, such as for example a neuronal network. Neuronal networks are particularly well suited, since they simulate the human brain as precisely as possible. The revised respirator data can thereby be ascertained with the aid of the sent or analysed respirator data, with the aid of the individual patient data, with the aid of the stored respirator data and/or with the aid of the known therapy data. This enables an independent compilation of respirator data in the at least one processing unit and the provision of the revised respirator data for therapies of different types of patients, such as for example asthmatic patients, apnoea patients or hypopnoea patients. This also enables the compilation of individual respirator data for therapy recommendations in the computing unit of the at least one processing unit, without a user, for example a member of the medical staff, having to carry out a correction of the analysed respirator data on the respirator.

Alternatively or in addition, at least one further self-learning computing algorithm based on at least one artificial intelligence is used in the computing unit, such as for example a simulation method, a phenomenological method or a symbolic network. The quality during the revision of the analysed respirator data can thus be further improved.

With the aid of the revised respirator data, at least one therapy recommendation with associated respirator data is advantageously compiled in the computing unit of the at least one processing unit, so that the patient to be ventilated can receive therapy without a user, for example a member of the medical staff, having to intervene or having to participate.

Following the transmission of the analysed and revised respirator data (step e)), a further revision of the analysed and revised respirator data preferably takes place on the at least one external receiving unit, so that for example the respirator data analysed and revised by the at least one processing unit can be adapted to empirical values of an external user.

Alternatively, following the transmission of the analysed and revised respirator data (step e)), a further revision of the analysed and revised respirator data takes place in the processing unit. Computing algorithms, for example, can be used for this, which function in a self-learning manner on the basis of neuronal networks and optimise the analysed respirator data in a straightforward and rapid manner.

Alternatively, following the transmission of the analysed and revised respirator data (step e)), a further revision of the analysed and revised respirator data takes place on at least one external receiving unit and a further revision of the analysed and revised respirator data takes place in the processing unit. Computing algorithms and external empirical values can for example be combined with one another and the analysed and revised respirator data can be efficiently optimised.

A correction of individual respirator data advantageously takes place in the further revision of the analysed and revised respirator data, wherein the correction advantageously takes place in respect of individual stored respirator data, so that a rapid adaptation to empirical values takes place.

The further revision of the analysed and revised respirator data also advantageously takes place on the at least one external receiving unit by a user, so that for example the user's expertise can enter into the revision or into the correction of the analysed and revised respirator data.

A software application for the at least one external receiving unit or the at least one respirator is advantageously made available in the at least one memory of the processing unit. The user thus gains access with the at least one receiving unit to the software application and can for example always work with the up-to-date version of the software application, wherein the up-to-date version of the software application is sent, advantageously unrequested, to the at least one external receiving unit and then installed.

The transmission of the further revised respirator data preferably takes place from at least one external receiving unit to the at least one processing unit, so that the further revised respirator data is subsequently available to many users for various applications.

Alternatively, the transmission of the further revised respirator data takes place from the at least one processing unit to a communication module, so that the further processed respirator data are available to different respirators.

Alternatively, the transmission of the further revised respirator data takes place from the at least one external receiving unit to the at least one processing unit and furthermore the transmission of the further revised respirator data takes place from the at least one processing unit to a communication module, so that the further revised respirator data are available for example to a plurality of respirators in the communication network.

This communication module is advantageously the communication module of the at least one respirator, so that the further revised respirator data can subsequently be used directly in the respective respirator.

The further revised respirator data are advantageously stored in the at least one processing unit, so that the further revised respirator data can always be used for further intended uses.

The further revised respirator data are advantageously used for further analyses with further stored respirator data in the at least one processing unit. For example, respirators of an identical device type and respirators of a different device type can thus be compared in the at least one processing unit.

The transmission of the further revised respirator data preferably takes place from the at least one processing unit to the at least one further external receiving unit, so that for example further expertise of a user is brought into the further revised respirator data.

The transmission of the further revised respirator data advantageously takes place from the at least one processing unit to the at least one further external receiving unit in order to alert a user, wherein rapid and direct action, for example direct adjustment of the respirator data, can take place on the at least one respirator by the user.

At least one input is advantageously carried out by a user or by the patient on an input unit of the at least one respirator, so that the user or the patient can input the respirator data, such as for example patient data, ambient conditions at the location or individual messages, into the at least one respirator. The latter can be further processed in the at least one respirator.

The at least one input is advantageously sent to the at least one processing unit, so that the input be used independently of the at least one respirator.

The at least one input is advantageously used in the at least one processing unit for the revision of the respirator data, so that the input created by the user or patient can be used directly in the at least one processing unit.

The at least one input is also advantageously transmitted to at least one external receiving unit, so that an external user acquires access to the input and the input can be monitored by this external user, for example by a member of the medical staff.

Following the analysis of the sent respirator data (step c)), in the event of a range deviation of at least one of the analysed respirator data from a safety range previously defined for the purpose, an alarm signal is preferably transmitted to at least one external receiving unit, so that an automatic alert is implemented.

Following the analysis of the sent respirator data (step c)), in the event of a range deviation of at least one of the analysed respirator data from a safety range previously defined for the purpose, an alarm signal is alternatively transmitted to at least one communication module, so that an alarm signal is subsequently displayed at the location of the at least one respirator.

Following the analysis of the sent respirator data (step c)), in the event of a range deviation of at least one of the analysed respirator data from a safety range previously defined for the purpose, an alarm signal is alternatively transmitted to at least one external receiving unit and an alarm signal is transmitted to at least one communication module, so that an alert is simultaneously implemented at a number of locations and for different users and the safe operation of the at least one respirator is thus guaranteed.

The alarm signal is advantageously transmitted to the communication module of the at least one respirator, so that an alert takes place directly on the respirator concerned.

Following the analysis of the sent respirator data (step c)), in the event of a range deviation of at least one of the analysed respirator data from a safety range previously defined for the purpose, an alarm signal is advantageously transmitted from the at least one processing unit, so that a simple alarm system is implemented which has an extensive range, since the alert is emitted centrally from the at least one processing unit.

Following the revision of the analysed respirator data (step d)), in the event of a range deviation of at least one of the revised respirator data from a safety range previously defined for the purpose, an alarm signal is alternatively or additionally transmitted to at least one external receiving unit, so that an automatic alert is implemented.

The analysis of the sent respirator data in the at least one processing unit (step c)) preferably includes an evaluation of the sent respirator data in the computing unit of the at least one processing unit with further respirator data, so that a comprehensive respirator data analysis can be carried out in the computing unit of the at least one processing unit. In the evaluation, individual respirator data points or entire respirator data point curves are compared with one another and put into a relationship, so that the evaluated respirator data can be presented in an understandable way to a user or a patient.

The sent respirator data are advantageously evaluated in relation to historical respirator data, so that for example long-term test data enter into the evaluation of the sent respirator data.

These historical respirator data may be university and clinical studies. These historical respirator data may also be respirator data approved by different state organisations or approved therapy recommendations. When respirators are operated differently in different countries and climatic zones, these respirator data can also be stored as historical respirator data and can thus contribute to the evaluation of the sent respirator data. Mechanical respirator data from various device manufacturers can for example also be stored as historical respirator data, which contribute to the evaluation or to the synchronisation of the sent respirator data.

The sent respirator data are particularly advantageously evaluated in relation to historical respirator data of the at least one respirator data, so that for example a wear-and-tear trend of different components in the respirator is determined.

With the aid of the evaluated respirator data, at least one therapy recommendation with associated respirator data is preferably compiled in the at least one processing unit, so that the patient to be ventilated can receive therapy without a user, for example a member of the medical staff, having to intervene.

The at least one therapy recommendation and the associated respirator data are advantageously transmitted to a communication module, so that the therapy recommendation and the associated respirator data are transmitted to the at least one respirator and can be used therein.

The at least one therapy recommendation and the associated respirator data are advantageously transmitted to the communication module of the at least one respirator, so that the therapy recommendation and the associated respirator data are transmitted to and used in the respirator which provides the basis for the therapy recommendation and the associated respirator data.

The at least one therapy recommendation and the associated respirator data are alternatively or additionally transmitted to at least one external receiving unit. The at least one therapy recommendation and the associated respirator data can thus be examined by an external user.

The evaluation of the respirator data in the at least one processing unit advantageously takes place on the basis of historical patient data, such as for example patient data from an anamnesis, additional parameters from external devices such as CO₂ and SpO₂ measurements. A medication anamnesis of the individual patient, for example, is taken into account in the respirator data, so that the at least one processing unit compiles therapy recommendations with regard to the type and dose of drugs and transmits the latter to the at least one respirator, so that the user of the at least one respirator can directly carry out the medicinal therapy for the patient to be ventilated. Alternatively or in addition, the therapy recommendations include the type and dose of new drugs for the patient to be ventilated.

The analysed and revised respirator data are preferably transmitted from the at least one processing unit to the respective device manufacturer of the at least one respirator. The respirators can thus be adjusted, calibrated or used for the further development of new respirator models by the device manufacturer using real-time respirator data.

The transmitted respirator data are preferably displayed on at least one display, so that the latter can easily be visualised.

The transmitted respirator data are advantageously made available, with the aid of an individual message, for the user of the at least one respirator, so that different users can be informed individually, for example in different languages.

The transmitted respirator data are alternatively displayed on a display and, with the aid of an individual message, made available to the user of the at least one respirator, so that the user can understand the transmitted respirator data in a personalised way and, depending on his or her skills, can react more quickly to the transmitted respirator data. The user-friendliness of the at least one respirator is thus improved.

The individual for the user advantageously includes therapy recommendations, which are directly matched to the at least one respirator and are based on the analysed and revised respirator data, evaluated respirator data or revised respirator data. The user of the respirator can thus act immediately and adapt the adjustments on the respirator, so that the patient to be ventilated can quickly be attended to.

Alternatively or in addition, the individual message for the user includes instructions concerning medication to be administered, wherein the instructions include for example the type of medication, the administration dose, the administration amount and the time for administration. Specialist instructions can thus be transmitted from specialist staff, for example a consultant specialising in asthmatic patients, to the user of the respirator.

The transmitted respirator data are advantageously displayed on a display of the at least one respirator, so that these respirator data can be seen by a user directly on the at least one respirator.

The transmitted respirator data are particularly preferably displayed with the aid of a graphic representation on a display, so that for example the analysed respirator data and the revised respirator data can be presented in an easily compared form.

The communication network preferably comprises a service system unit, wherein the analysed and revised respirator data are sent from the at least one processing unit to the service system unit. A billing system, for example, can thus be integrated into the communication network. An examination of the user rights of various software applications on the at least one respirator or on the at least one external receiving unit can for example also are alternatively take place by means of the service system unit.

At least one further respirator is preferably operated in the communication network, so that a plurality of respirator data are available in the communication network.

The at least one further respirator is advantageously connected via a further communication module to the communication network, wherein the at least one further communication module sends further respirator data, i.e. from the corresponding respirator to the at least one processing unit. The analysis and revision of the sent respirator data in the at least one processing unit is thus improved on account of the increased number of respirator data.

The at least one further respirator is advantageously connected via the one communication module, which also connects the at least one respirator to the communication network, wherein the subsequent sending of the transmitted respirator data can be in a pooled form.

The sent respiratory data of at least two respirators are preferably analysed in the at least one processing unit, so that the device performance of the two respirators can be compared directly and in real time.

The sent respirator data from at least two respirators are advantageously compared in the at least one processing unit with historical respirator data. A statistical evaluation of a large amount of respirator data, for example, is thus enabled.

The computer program product according to the invention, which recognises a communication module connected to the at least one respirator after is has been loaded in a memory of the at least one processing unit using a receiving unit of the processing unit, then puts into effect one of the previously described methods for operating at least one respirator in a communication network. The at least one respirator can thus be operated automatically and simply in the communication network.

Further advantages, features and details of the invention emerge from the following description, in which examples of embodiment of the invention are described by reference to the drawings.

### BRIEF DESCRIPTION OF THE DRAWING

The list of reference numbers, as also the technical content of the claims and figures, is a component part of the disclosure. The figures are described in a coherent and comprehensive manner. Identical reference numbers denote identical components, reference numbers with different indices indicate functionally identical or similar components.

In the figures:

FIG. 1 shows a first graphic representation of the method according to the invention for operating a respirator in a communication network,

FIG. 2 shows a further graphic representation of the method according to the invention for operating a plurality of respirators in a communication network,

FIG. 3 shows a further graphic representation of the method according to the invention for operating a plurality of respirators in a communication network, wherein the respirator data are also sent to the respective device manufacturer,

FIG. 4 shows a further graphic representation of the method according to the invention for operating a plurality of respirators in a communication network, wherein the respirator data can also be analysed with historical respirator data,

FIG. 5 shows a further graphic representation of the method according to the invention for operating a plurality of respirators in a communication network, wherein the respirator data are also sent worldwide to different users,

FIG. 6 shows a method according to the invention for operating a plurality of respirators in a communication network represented in a flow chart, and

FIG. 7 shows a method according to the invention for operating a plurality of respirators in a communication network, wherein the processing unit comprises a warning system.

### DETAILED DESCRIPTION

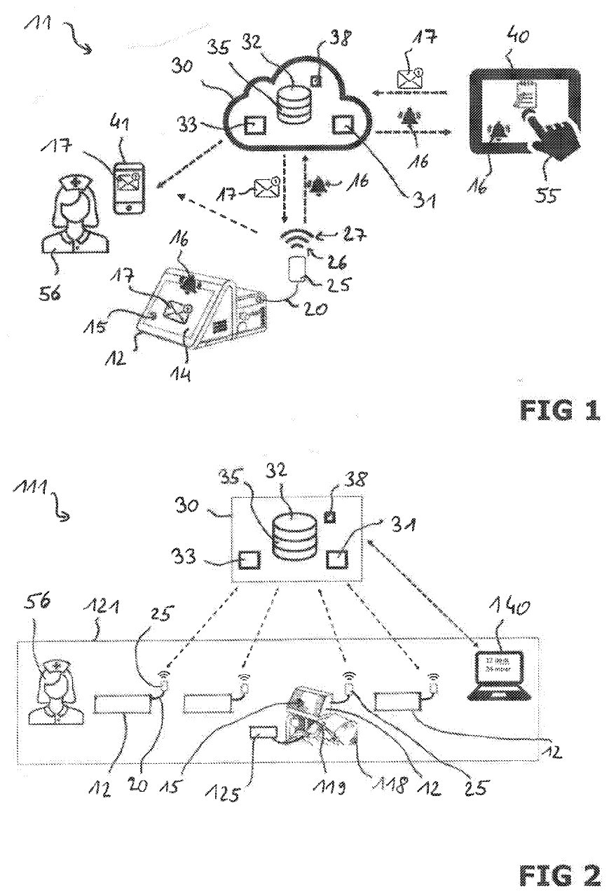

FIG. 1 shows a communication network 11 with a respirator 12, which is connected via a cable connection 20 to an external communication module 25. In an alternative embodiment, communication module 25 can be integrated in respirator 12 and electronically connected internally to respirator 12. Communication module 25 comprises a sending device 26 for sending respirator data, alarm signals 16 and messages 17 as well as a receiving device 27 for receiving the respirator data, alarm signals 16 and messages 17. The respirator data are on the one hand data of respirator 12 which are previously measured with a patient to be ventilated and transmitted from the sensor unit required for this to respirator 12, or data of respirator 12 which relate to respirator 12 itself. Respirator 12 comprises an internal and/or an external sensor unit, wherein the sensor unit measures for example ventilation-relevant parameters and vital parameters, such as for example body temperature, blood pressure, pulse and/or the ECG of the patient to be ventilated, which are transmitted to respirator 12 and/or to communication module 25. These include for example technical performance data which contain the mechanical, pneumatic and electrical performance of respirator 12. Communication module 25 sends and receives respirator data, alarm signals 16, messages 17, wherein the latter are sent as standardised or as an individual message. The respirator data are picked up from respirator 12 and, in a first step (step a)), are transmitted with the aid of cable connection 20 to communication module 25. Communication module 25 is designed in such a way that it receives respirator data independently of device manufacturer 50 of respirator 12 and then processes the latter, in such a way that they are sent in a further step (step b)) in an arbitrary data format, here via GSM standard, to processing unit 30. Moreover, further transmission standards, such as for example Bluetooth® or Wireless LAN are possible. It is advantageous if respective communication module 25 encodes the respirator data to be sent in advance and decodes the received, revised respirator data, so that sufficiently high security is guaranteed in communication network 11. Processing unit 30 comprises a receiving unit 31 for receiving the sent or transmitted respirator data, wherein these respirator data are stored in a memory 35 of processing unit 30. Processing unit 30 further comprises a computing unit 32, in which the respirator data are analysed in a further step (step c)). The analysed respirator data in computing unit 32 are then revised with the aid of a self-learning computing algorithm (step d)). The self-learning computing algorithm is based on an artificial intelligence, which is constituted as a neuronal network. In the neuronal network, revised respirator data are ascertained with the aid of the sent, analysed, evaluated or further revised respirator data, the individual patient data, the stored respirator data and/or known therapy data.

The analysed and revised respirator data are then transmitted by a sending unit 33 of processing unit 30 to communication module 25 and from there are relayed to respirator 12. The analysed and revised respirator data can also be transmitted simultaneously or subsequently by sending unit 33 of processing unit 30 to an external receiving unit 40 (step e)). For example, external receiving unit 40 comprises a smart phone, a tablet or a computer. For the transmission, external receiving unit 40 comprises a sending and a receiving device (not shown). In a further step, the transmitted respirator data are used (step f)).

The analysed and revised respirator data are picked up by a user 55 at external receiving unit 40, are for example commented on and, if appropriate, further revised. Individual messages 17 and/or an alarm signal 16 can thus be transmitted, wherein message 17 contains for example an instruction for another user 56 of respirator 12. Furthermore, it is possible for users 55 and 56 to correct respirator data with a software application installed on external receiving unit 40, for example to change individual analysed respirator data or respirator data points or to correct or further revise entire curves of respirator data or respirator data points. Alternatively or in addition to this, computing unit 32 of processing unit 30 is designed to change individual analysed respirator data or to correct or revise entire curves of respirator data. Computing unit 32 of processing unit 30 recognises a faulty operation of respirator 12 by user 56.

Processing unit 30 compiles a therapy recommendation with associated respirator data with the aid of the evaluated and/or with the aid of the revised respirator data and/or with the aid of the further revised respirator data. The evaluation of the respirator data in processing unit 30 is based on historical patient data, such as for example patient data from an anamnesis. A medication anamnesis of the individual patient, for example, is taken into account in the respirator data, so that processing unit 30 compiles therapy recommendations with regard to the type and dose of drugs or new drugs. The latter are transmitted to respirator 12, so that user 56 of respirator 12 can directly carry out the medicinal therapy of the patient to be ventilated.

The analysed and revised or evaluated respirator data are then transmitted via processing unit 30 back to communication module 25 of respirator 12 and relayed by means of cable connection 22 to respirator 12. Respirator 12 contains a software application, with which the transmitted respirator data are retrieved and, if appropriate, further revised or corrected. The software application, which is assigned to respective respirators 12, is stored in memory 35 of processing unit 30, wherein a new version of the software application is regularly and, optionally unrequested, installed on external receiving units 40 and on respective respirators 12. A receiving unit, which can store and also execute the software application, can serve as external receiving unit 40.

The transmitted respirator data are used in respirator 12 for the adjustment of respirator 12 or for alerting user 56 of respirator 12, for example with the aid of an alarm signal 16 or a message 17. For better visualisation of the respirator data, alarm signal 16 and, if appropriate, message 17 is displayed on a display 14 of respirator 12. Respirator 12 comprises an input unit 15 for inputting an input by user 56 or by the patient, which input unit is arranged on respirator 12 or is designed as a touch screen in display 14. Alternatively, alarm signal 16 and/or message 17 can also be displayed on a further display. Moreover, processing unit 30 transmits alarm signal 16 or message 17 to a further external receiving unit 41, which is assigned to a further user 56, who can then take targeted action, for example directly at the location of the given respirator 12 involved. Alternatively, communication module 25 sends alarm signal 16 and/or message 17 directly to further external receiving unit 41.

Computing unit 32 of processing unit 30 recognises in the analysis of the sent and stored respirator data whether a range deviation of one of the analysed or revised respirator data from a previously defined safety range is taking place. For this purpose, safety ranges are previously defined for all respirator data and stored in memory 35 of processing unit 30, for example the range of the permitted, very critical, inhalation pressure. In the event of a range deviation of one of the analysed or revised respirator data, an alarm signal 16 is transmitted from processing unit 30 to external receiving units 40 and 41 and to communication module 25 concerned and is also relayed to respirator 12.

Processing unit 30 also comprises a service system unit 38, to which respirator data are sent. Service system unit 38 comprises a billing system (not shown), which is integrated in communication network 11. A revision of user rights of various software applications on respirator 12 or on external receiving units 40 and 41 can thus be monitored and automatically charged. Different user rights of respective respirators 12 from different device manufacturers can also be monitored in communication network 11.

FIG. 2 shows an additional or alternative embodiment of the method according to the invention with a plurality of respirators 12 and a plurality of external communication module 25 in a communication network 111. The plurality of respirators 12 are located in a confined area 121, for example in a hospital, and communicate with processing unit 30. Processing unit 30 is thus designed to analyse and to revise the respirator data from a plurality of respirators 12 and to transmit the latter to external communication modules 25 and to a receiving unit 140 distant from respirators 12. The respirator data are on the one hand data of respirator 12 which are previously measured for a patient 118 to be ventilated and transmitted from sensor unit 119 required for this to respirator 12, or data which relate to respirator 12 itself. Respirator 12 comprises an internal or external sensor device 125, wherein sensor device 125 measures ventilation-relevant parameters and vital parameters, which are transmitted to respirator 12 and/or to communication module 25. The detected respirator data are then used in confined area 121.

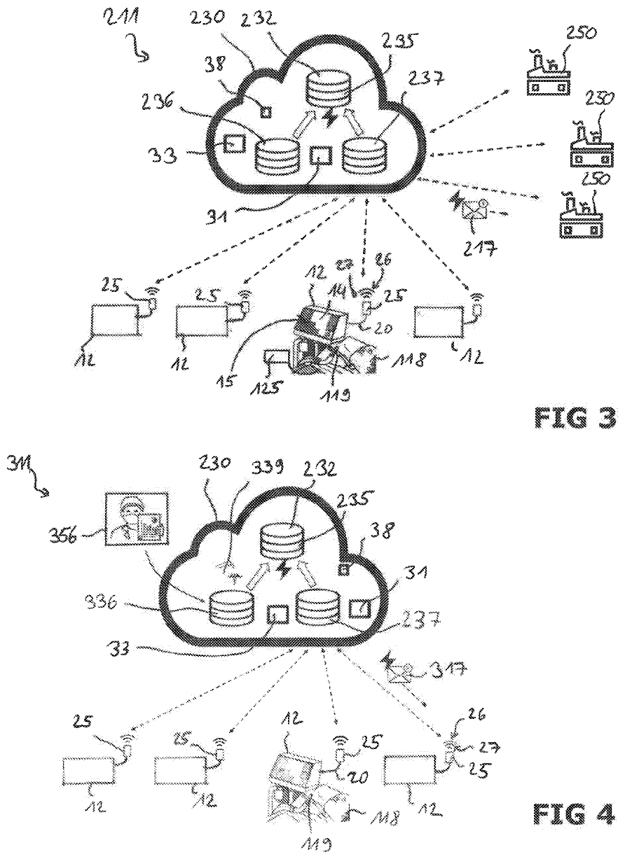

FIG. 3 shows an additional or alternative embodiment of the method according to the invention with a plurality of respirators 12 and a plurality of external communication modules 25 in a communication network 211. The respirator data are sent from a plurality of respirators 12 by means of the plurality of communication modules 25 to processing unit 230, which is basically similar to processing unit 30, and are stored there in memory 235. A further memory 236 is arranged in processing unit 230, which stores for example historical respirator data from respirators 12 from different device manufacturers 250, for example in the given language of device manufacturer 250. The sent respirator data are thus sorted according to the medical and/or safety-related relevance and stored in respective memories 235, 236. For this purpose, processing unit 230 contains, apart from illustrated memories 235 and 236, any number of further memories 237. The sent and stored respirator data are analysed and compared in computing unit 232 of processing unit 230 with the respective historical respirator data and transmitted to respective device manufacturers 250. Device manufacturer 250 receives a message 217, for example concerning a malfunction in respirator 12, or receives the analysed and revised respirator data directly. Device manufacturers 250 then use the respirator data transmitted to them, for example to develop new respirator modes or to improve the existing respirator modes. Device manufacturers 250 then transmit the further revised respirator data to processing unit 230. The further revised respirator data are then stored in respective memories 235, 236 and, if appropriate, transmitted to respective communication modules 25 and relayed to respective respirators 12.

FIG. 4 shows a further additional or alternative embodiment of the method according to the invention with a plurality of respirators 12 and a plurality of external communication modules 25 in a communication network 311. Further memory 336 of processing unit 230 contains historical respirator data, which result for example from clinical studies 339 and are stored sorted there. These historical respirator data have been or are generated by a user 356, for example an external team of doctors. The respective respirator data are stored according to their medicinal relevance, so that the latter can then be used, depending on the intended purpose of the respirator data, for an analysis with the sent respirator data. The sent and stored respirator data are analysed and revised or compared in computing unit 232 of processing unit 230 with the respective historical respirator data and are then transmitted again to respective communication modules 25 of respirators 12, then relayed to respective respirators 12 and used in respective respirator 12. Messages 317 can also be transmitted to respirators 12.

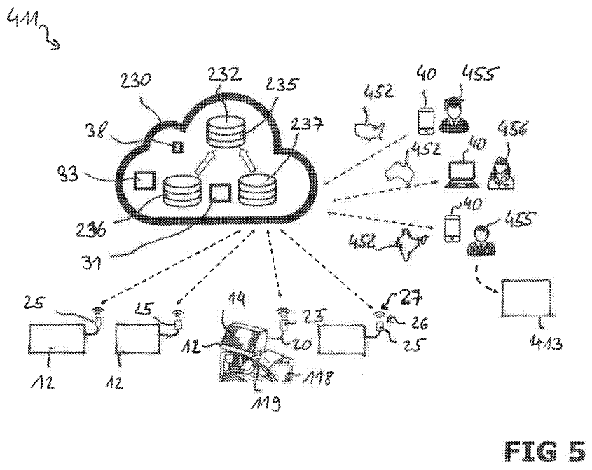

FIG. 5 shows a further additional or alternative embodiment of the method according to the invention with a plurality of respirators 12 and a plurality of external communication modules 25 in a communication network 411. The analysed and revised or compared respirator data in processing unit 230 are used to inform or to train users 455 of respirators 12 worldwide. The analysed and revised respirator data are transmitted from processing unit 230 to individual external receiving units 40, which are then used there by respective user 455 or 456. Users 455 will use the respirator data depending on the territorial location 452 of respirator 12. The transmitted respirator data are used by user 455 for training purposes or for monitoring an individual respirator 12.

Moreover, a user 455 uses the analysed and revised respirator data for example for manual input on a respirator 413, which is not directly integrated in communication network 411.

Users 455 and 456 can further revise the transmitted respirator data with the aid of the software application and then, with the aid of their external receiving units 40, transmit the latter back to processing unit 230. These further revised respirator data are then stored and, if appropriate, relayed onward to respective communication modules 25 and to respirators 12.

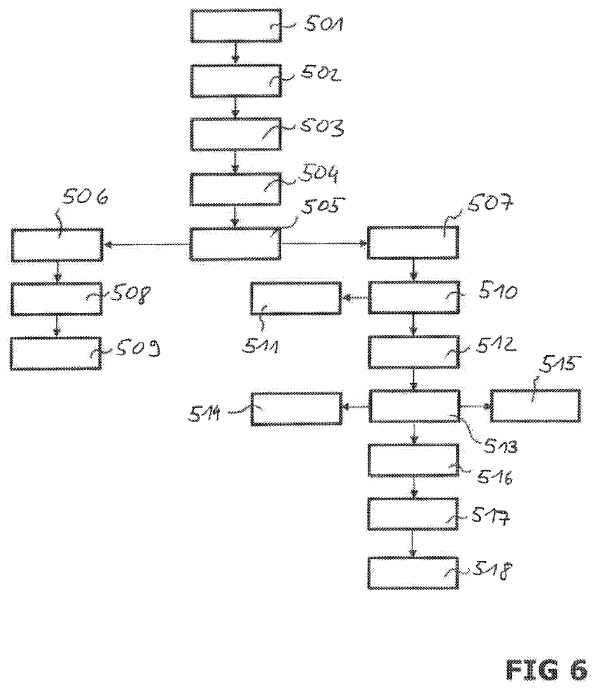

FIG. 6 shows the method according to the invention for operating a respirator 12 in one of the aforementioned communication networks 11, 111, 211, 311 and 411 in a flow chart. The process steps are similar to the previously described embodiments (FIG. 1 to FIG. 5) of a plurality of respirators 12 and can be carried out simultaneously or offset in time and accordingly adapted.

In a first step 501, respirator data are transmitted from respirator 12 to communication module 25 and processed there (step 502). Communication module 25 sends the respirator data to processing unit 30 (step 503), wherein the respirator data are stored there (step 504) and analysed and revised in computing unit 32. For example, these respirator data are compared with already stored respirator data (step 505) and/or revised with the aid of the neuronal network.

The analysed and revised respirator data are then transmitted to a communication module 25 and/or to an external receiving unit 40 and 41 (step 506), wherein the transmitted respirator data are used there (step 507), for example on receiving unit 40 with the aid of a software application. The transmitted respirator data are on the one hand transmitted from a communication module 25 to a respirator 12 (step 508) and, as required, displayed on display 14 of respirator 12 (step 509) or used, for example further processed, by a user 55 or 56 on receiving unit 40 (step 510). The further revised respirator data are then transmitted from receiving unit 40 or 41 directly to communication module 25 and from there relayed to the respirator (step 511) or transmitted from receiving unit 40 to processing unit 30 (step 512) and stored there in memory 35 (step 513).

The revised respirator data are then either transmitted to a further receiving unit 41 of a further user 56 (step 514) and/or for example transmitted to the respective device manufacturer 250 (step 515). The stored and further revised respirator data are then transmitted again to a communication module 25 (step 516) and relayed from there to respirator 12 (step 517), where the further revised respirator data are used (step 518).

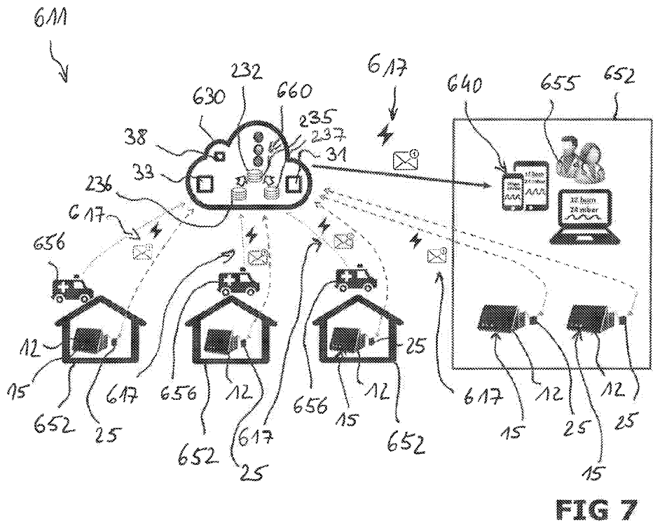

FIG. 7 shows a further additional or alternative embodiment of the method according to the invention with a plurality of respirators 12 and a plurality of external communication modules 25 in a communication network 611. The plurality of respirators 12 are used for the ventilation of patients, who are located at different locations 652, for example in their homes or also in individual hospitals, and do not therefore require permanent monitoring by a user 656, for example a nurse.

In order to ensure regular checks on the individual patients or respective respirators 12, a check on the patients by users 656 is carried out at regular intervals.

Respirators 12 or their communication modules 25 send the respirator data to processing unit 630, wherein this processing unit 630 is comparable to the previously shown processing units 30, 230. Furthermore, inputs are inputted by the patient or user 656, for example replies to questionnaires as to the patient's well-being or the status of the respirator, at input unit 15 of respective respirators 12, which inputs are also sent to processing unit 630. The inputs include respirator data, such as for example patient data, ambient conditions at the location or individual messages 617. The inputted respirator data are analysed and/or revised and/or evaluated in processing unit 630. For example, the input is used in computing unit 32, 232 of the at least one processing unit 630 for the further revision of the respirator data.

Processing unit 630 comprises a warning system 660, which classifies the analysed and/or revised and/or further revised and/or evaluated respirator data into three categories, depending on their medical and/or safety-related relevance. Depending on their category, the respirator data and/or individual messages are transmitted to external receiving units 640 of users 655, so that the latter are informed about the condition of the patient and/or the status of individual respirators 12 and, if need be, can intervene in the ventilation process of the patient.

Warning system 660 includes for example a traffic-light system, wherein the first category includes respirator data which indicate a problem-free condition of the patient to be ventilated or of respective respirator 12. The second category includes for example respirator data which require action on the part of user 656 or on the part of the given patient, for example repositioning of a breathing mask on the patient. The third category includes for example critical ventilation data, which requires immediate intervention by users 655 or 656, such as for example a safety range of individual respirator data being exceeded.

Processing unit 30, 230, 630 also comprises a computer program product, which recognises communication module 25 connected to respirator 12 after it has been loaded into memory 35, 235 of processing unit 30, 230, 630 using a receiving unit 31 of processing unit 30, 230, 630, and then puts into effect the present method for operating at least one respirator in a communication network 11, 111, 211, 311, 411, 611 as previously in the described embodiments (FIG. 1 to FIG. 7).

### LIST OF REFERENCE NUMBERS

11 Communication network
12 Respirator
14 Display of 12
15 Inputting unit of 12
16 Alarm signal
17 Message
20 Cable connection
25 Communication module
26 Sending device of 25
27 Receiving device of 25
30 Processing unit
31 Receiving unit of 30
32 Computing unit of 30
33 Sending unit of 30
35 Memory of 30
38 Service system unit
40 Receiving unit
41 Further receiving unit
55 User
56 Further user
111 Further communication network
118 Patient
119 Sensor unit
121 Confined area
125 Sensor device
140 Receiving unit
211 Further communication network
217 Message
230 Processing unit
232 Computing unit of 230
235 Memory of 230
236 Memory of 230
237 Memory of 230
250 Device manufacturer
311 Further communication network
317 Message
336 Memory of 230
339 Study
356 User
411 Further communication network
413 Respirator (not integrated in 411)
452 Location
455 User
456 Further user
501-518 Process steps
611 Further communication network
617 Message
630 Processing unit
640 Receiving unit
652 Location
655 User
656 User
660 Warning system

1. A method for operating a respirator (12) in a communication network (11; 111; 211; 311; 411; 611), wherein the communication network (11; 111; 211; 311; 411; 611) comprises at least one processing unit (30; 230; 630), a cloud, and the at least one processing unit (30; 230; 630) comprises a computing unit (32; 232), and the at least one respirator (12) is connected via a communication module (25) to the communication network (11; 111; 211; 311; 411; 611), wherein the method comprises the following steps:
a) transmitting respirator data from the at least one respirator (12) to the communication module (25);
b) sending of the transmitted respirator data from the communication module (25) to the at least one processing unit (30; 230; 630), wherein the sending advantageously takes place using a GSM standard;
c) analyzing the sent respirator data in the at least one processing unit (30; 230; 630)
d) revising the analysed respirator data in the computing unit (32; 232) of the at least one processing unit (30; 230; 630);
e) transmitting the analysed and revised respirator data from the at least one processing unit (30; 230; 630) to a communication module, wherein this communication module is advantageously the communication module (25) of the at least one respirator (12), and/or transmission of the analysed and revised respirator data from the at least one processing unit (30; 230; 630) to at least one external receiving unit (1, 41; 140; 640); and
f) using the transmitted respirator data.

2. The method according to claim 1, wherein, after step e), the analysed and revised respirator data are transmitted from the communication module (25) of the at least one respirator (12) to the at least one respirator (12).

3. The method according to claim 1, wherein, before step d), the sent respirator data are stored in the at least one processing unit (30; 230; 630), advantageously stored sorted according to their medical and/or safety-related relevance, and are also advantageously stored in different memories (35; 235, 236, 237; 336).

4. The method according to claim 1, wherein the analysed and revised respirator data are classified into at least three categories, advantageously depending on their medical and/or safety-related relevance, and are advantageously transmitted to at least one external receiving unit (40, 41; 140; 640).

5. The method according to claim 1, wherein the at least one communication module (25) previously processes, preferably encodes, the transmitted respirator data for secure sending to the at least one processing unit (30; 230; 630), and also advantageously processes, advantageously decodes, the transmitted respirator data for secure relaying to the respirator (12).

6. The method according to claim 1, wherein the revision of the analysed respirator data in step d) in the computing unit (32; 232) takes place with the aid of at least one computing algorithm, advantageously with at least one self-learning computing algorithm.

7. The method according to claim 1, wherein after step e), a further revision of the analysed and revised respirator data, preferably a correction of individual analysed and revised respirator data, takes place at at least one external receiving unit (40, 41; 140; 640), also advantageously by a user (55, 56; 356; 455, 456; 655, 656) and/or in the processing unit (30; 230; 630).

8. The method according to claim 7, wherein the transmission of the further revised respirator data takes place from at least one external receiving unit (40, 41; 140; 640) to the at least one processing unit (30; 230; 630) and/or the transmission of the further revised respirator data takes place from the at least one processing unit (30; 230; 630) to a communication module, wherein this communication module is the communication module (25) of the at least one respirator (12).

9. The method according to claim 7, wherein the further revised respirator data are stored in the at least one processing unit (30; 230; 630) and are advantageously used for further analyses with further stored respirator data in the at least one processing unit (30; 230; 630).

10. The method according to claim 7, wherein the transmission of the further revised respirator data takes place from the at least one processing unit (30; 230; 630) to at least one further external receiving unit (40, 41; 140; 640), advantageously to alert a user (55, 56; 356; 455, 456; 655, 656).

11. The method according to claim 1, wherein at least one input is carried out by a user (55, 56; 356; 455, 456; 655, 656) or by a patient (118) on an input unit (15) of the at least one respirator (12) and the at least one input is advantageously sent to the at least one processing unit (30; 230; 630), wherein the at least one processing unit (30; 230; 630) also advantageously uses the at least one input for the revision of the respirator data, and also advantageously the at least one input is transmitted to at least one external receiving unit (40, 41; 140; 640).

12. The method according to claim 1, wherein, after step c), in the event of a range deviation of at least one of the analysed respirator data and/or at least one of the revised respirator data from a safety range previously defined for the purpose, an alarm signal (16) is transmitted, advantageously by the at least one processing unit (30; 230; 630), to at least one external receiving unit (40, 41; 140; 640) and/or to at least one communication module, wherein this communication module is advantageously the communication module (25) of the at least one respirator (12).

13. The method according to claim 1, wherein the analysis of the sent respirator data in the at least one processing unit (30; 230; 630) (step c)) includes an evaluation of the sent respirator data in the computing unit (32; 232) of the at least one processing unit (30; 230; 630) with further respirator data, advantageously in relation to historical respirator data, particularly advantageously of the at least one respirator (12).

14. The method according to claim 13, wherein, with the aid of the evaluated respirator data, at least one therapy recommendation with associated respirator data is compiled in the at least one processing unit (30; 230; 630) and the at least one therapy recommendation and the associated respirator data are advantageously transmitted to a communication module, advantageously to the communication module (25) of the at least one respirator (12) and/or to at least one external receiving unit (40, 41; 140; 640).

15. The method according to claim 13, wherein the evaluation of the respirator data in the computing unit (32; 232) of the at least one processing unit (30; 230; 630) takes place on the basis of historical patient data.

16. The method according to claim 1, wherein the analysed and revised respirator data are transmitted from the at least one processing unit (30; 230; 630) to the respective device manufacturer (250) of the at least one respirator (12).

17. The method according to claim 1, wherein the transmitted respirator data are displayed on a display, advantageously on a display (14) of the at least one respirator (12), and particularly advantageously with the aid of a graphic representation, and/or are made available by means of an individual message (17; 217; 317; 617) to the user (55, 56; 356; 455, 456; 655, 656) of the at least one respirator (12).

18. The method according to claim 1, wherein the communication network (11; 111; 211; 311; 411; 611) comprises a service system unit (38) and the analysed and revised respirator data are sent from the at least one processing unit (30; 230; 630) to the service system unit (38).

19. The method according to claim 1, wherein at least one further respirator (12) is operated in the communication network (11; 111; 211; 311; 411; 611), and the at least one further respirator (12) is connected, advantageously via a further communication module (25), to the communication network (11; 111; 211; 311; 411; 611), wherein the at least one further communication module (25) sends further respirator data to the at least one processing unit (30; 230; 630).

20. The method according to claim 1, wherein the sent respirator data of at least two respirators (12) are analysed and revised in the at least one processing unit (30; 230; 630), and are also advantageously compared with historical respirator data.
